(19) United States
(12) Patent Application Publication
(10) Pub. No.: US 2008/0312699 A1
(43) Pub. Date: Dec. 18, 2008

(54) RECESSED PLATE SYSTEM

(76) Inventors: Jeffrey Johnson, Brandon, MS (US); Pat Barrett, Jackson, MS (US)

Correspondence Address: CERMAK KENEALY & VAIDYA LLP, 515 E. BRADDOCK RD, SUITE B, ALEXANDRIA, VA, 22314, US

(21) Appl. No.: 12/101,431

(22) Filed: Apr. 11, 2008

Related U.S. Application Data
(60) Provisional application No. 60/911,114, filed on Apr. 11, 2007.

(51) Int. Cl. A61B 17/80; A61F 2/44; A61B 19/00

(52) U.S. Cl. 606/280; 623/17.11; 128/898

(57) ABSTRACT

A plate is positioned in an intervertebral space to inhibit or prevent an intervertebral spacer from backing out from the intervertebral space.

This application claims priority under 35 U.S.C. § 119 to provisional U.S. patent application No. 60/911,114, filed 11 Apr. 2007, the entirety of which is incorporated by reference herein.

### BACKGROUND

1. Field of Endeavor

The present invention relates to devices, systems, and processes useful in spinal surgery, and more specifically to disc replacement surgeries.

2. Brief Description of the Related Art

During vertebral disc replacement surgery, it is commonplace to insert an intervertebral spacer between two adjacent vertebrae, in the place of a ruptured or diseased disc. Such intervertebral spacers include bone grafts, peek cages, titanium cages, stainless steel cages, bioresorbable cages, and the like. Currently commercially available vertebral plates on the market are attached to the vertebral body on the outside of the vertebrae, which can cause damage to the adjacent blood vessels and even death of the patient.

There remains a need for an intervertebral plate system that can retain an intervertebral spacers in situ which does not suffer from the deficiencies of prior plates.

### SUMMARY

According to a first aspect of the invention, a system comprises an intervertebral spacer configured and arranged to be positioned between two vertebrae of a patient, and a plate configured and arranged to at least inhibit the intervertebral spacer from backing out when positioned between the two vertebrae of a patient.

According to another aspect of the present invention, a method comprises positioning an intervertebral spacer in an intervertebral space between two vertebrae of a patient, and positioning a plate at least partially in said intervertebral space and adjacent to the spacer, to at least inhibit the intervertebral spacer from backing out from said space.

Still other aspects, features, and attendant advantages of the present invention will become apparent to those skilled in the art from a reading of the following detailed description of embodiments constructed in accordance therewith, taken in conjunction with the accompanying drawings.

### BRIEF DESCRIPTION OF THE DRAWINGS

The invention of the present application will now be described in more detail with reference to exemplary embodiments of the apparatus and method, given only by way of example, and with reference to the accompanying drawings, in which:

FIG. 1 illustrates a side view of an exemplary device of the present invention installed between two vertebrae and adjacent to an interbody spacer;

FIG. 2 illustrates a front view of the device of FIG. 1;

FIG. 3 illustrates a top view of the device of FIG. 1 in situ;

FIG. 4 illustrates a cross-sectional side view of an exemplary device;

FIG. 5 illustrates a front view of a second exemplary embodiment;

FIG. 6 illustrates a cross-sectional side view of the device of FIG. 5;

FIG. 7 illustrates a front view of the device of FIG. 4;

FIG. 8 illustrates a cross-sectional view, taken at line A-A in FIG. 7;

FIG. 9 illustrates a cross-sectional view, taken at line B-B in FIG. 7;

FIG. 10 illustrates a cross-sectional side view of yet another exemplary embodiment;

FIG. 11 illustrates a top front perspective view of the device of FIG. 10; and

FIG. 12 illustrates a front view of the devices of FIG. 10.

### DETAILED DESCRIPTION OF EXEMPLARY EMBODIMENTS

Referring to the drawing figures, like reference numerals designate identical or corresponding elements throughout the several figures.

“Intervertebral Spacers” or “Interbody Spacers” are known per se, and a familiarity with their structures and functions is both well known to the skilled artisan and presumed herein; accordingly, details of Interbody Spacers will not be provided in this description.

One aspect of the present invention includes the use of a plate in conjunction with one or more intervertebral spacers to inhibit, and advantageously prevent, the intervertebral spacer(s) from backing out and, thus, limit or prevent blood vessel damage. According to one of numerous principles of the present invention, a recessed plate is positioned between the vertebral bodies along with the intervertebral spacer, which will inhibit or prevent the intervertebral spacer from backing out. Additionally, the recessed plate is optionally, yet advantageously, positioned inside the vertebral space along with the intervertebral spacer, which addresses the problem of blood vessel damage. A recessed plate embodying principles of the present invention is positioned inside of the vertebral space, limiting or eliminating interference with and damage to the adjacent blood vessels.

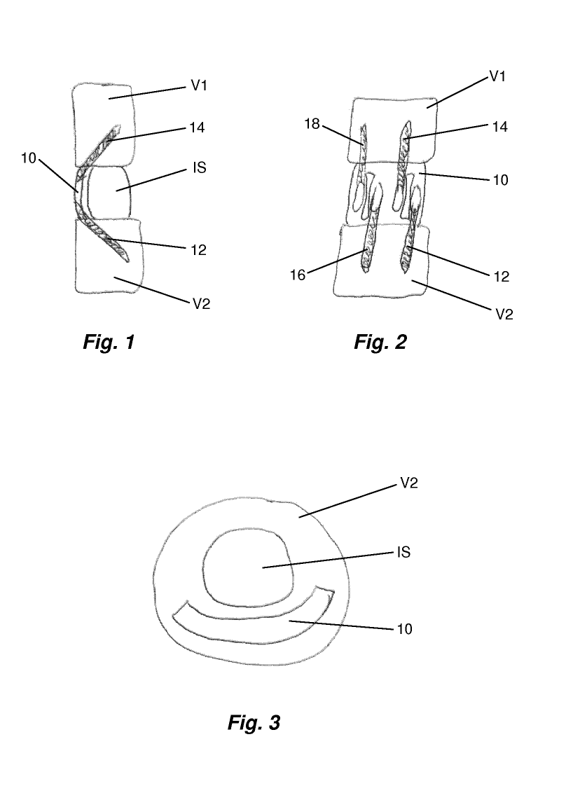

Turning now to the drawing figures, FIGS. 1-3 illustrate side, front, and top views, respectively, of a portion of a patient's body, including vertebrae, including an exemplary recessed plate 10 embodying principles of the present invention. The plate 10 is positioned between two vertebrae V1, V2 (which can be any vertebrae; their numbering is not limited to the first and second vertebrae) between which an interbody spacer IS has been installed. As discussed elsewhere herein, the interbody spacer IS can be any such spacer as is used in disc replacement surgeries, including, but not limited to, bone grafts, peek cages, titanium cages, stainless steel cage, bioresorbable cages, and the like. The plate 10 is held in place by at least two screws (four are illustrated) 12, 14, 16, 18, which pass through holes or openings in the plate and into the adjacent bone of the vertebrae V1, V2. As can be plainly seen from FIGS. 1 and 3, the plate 10 is positioned adjacent to and outside of the spacer IS, but advantageously, although not necessarily, does not extend beyond the outer surface of the vertebrae V1, V2. The plate 10, as with other exemplary embodiments described herein, keeps the interbody spacer IS from backing out from its position between the two vertebrae V1, V2, and is therefore sized and configured to fit between the two vertebrae.

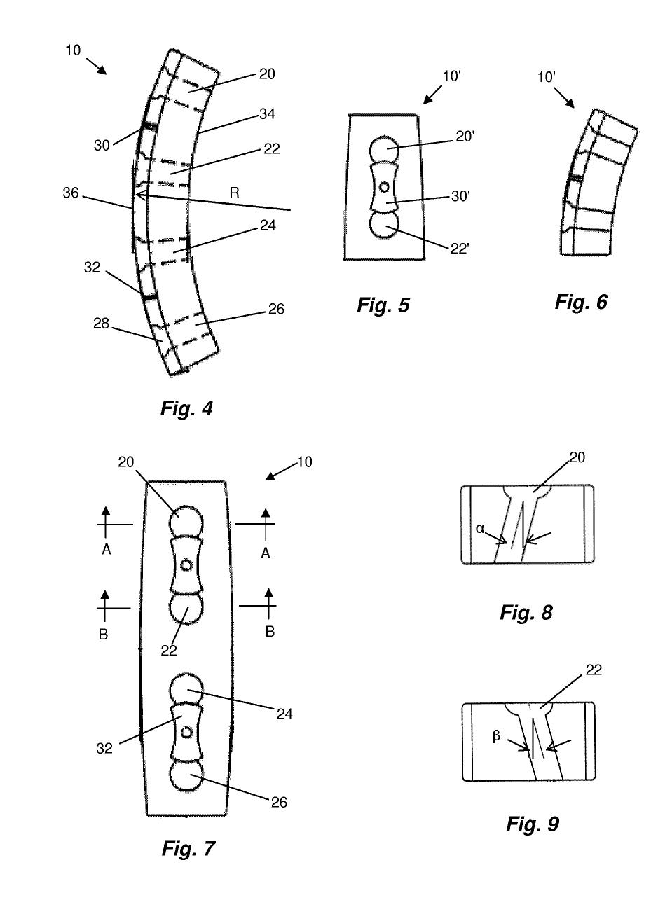

FIGS. 4 and 7 illustrate a first exemplary plate 10 embodying principles of the present invention. The plate 10 includes holes 20, 22, 24, 26, which extend through the body of the plate from a convex front surface 36 to a concave rear surface 34. By way of a non-limiting example, the radius of curvature of the plate 10, denoted R, is about 30.5 mm, although smaller and larger radii of curvature are also possible to better fit smaller and larger vertebrae, respectively. Each hole is configured to receive one of the screws 12, 14, 16, 18 therethrough, so that the plate 10 is firmly held in place when installed in the patient's body. The plate 10 also preferably includes a rotating lock 30, 32 for each screw, or optionally for multiple screws. Rotating locks usable in the present invention are described in U.S. Pat. Nos. 6,730,127 and 6,890,335, the entireties of both of which are incorporated by reference herein. The plate 10 is preferably curved to form convex and concave surfaces 34, 36; these curvatures can be on the same center of curvature or different centers, and, as discussed above, can have any radii of curvature that will permit the plate to function as described herein, including the same or different curvatures, or no curvature for either or both surfaces.

FIGS. 5 and 6 illustrate front and side cross-sectional views, respectively, of another exemplary embodiment 10′ which includes only two screw holes 20′, 22′, and a single lock 30′ to inhibit or prevent the screws (not illustrated) from backing out of the holes.

FIGS. 8 and 9 illustrate cross-sectional views, taken at lines A-A and B-B in FIG. 7, respectively, of the plate 10. Another optional, yet advantageous feature of a plate embodying principles of the present invention, is that the holes through the plate (holes 20, 22 are illustrated merely by way of example) extend at an angle α, β to the local normal direction of the plate, so that the screws will extend above and below the plate 10 when installed in a patient (see FIGS. 1 and 2). While those of skill in the art will easily be able to select particular useful angles α, β, from about 15° to about 75° is preferred; the angles α, β can be the same or different.

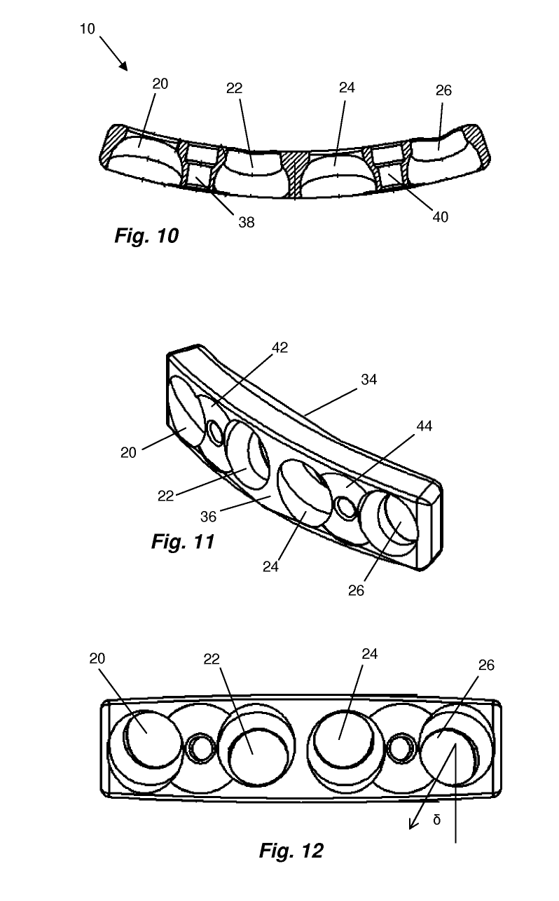

FIGS. 10-12 illustrate side cross-sectional, perspective, and front elevational views, respectively, of yet another embodiment of a plate 10 in accordance with principles of the present invention. In addition to the screw holes 20-26, each including a countersunk portion for each screw head, the plate includes bores 38, 40, to receive the locks previously described. Further optionally, the outer face of the plate can include slight recesses 42, 44, in which the lock is positioned and can rotate. FIGS. 11 and 12 in particular illustrate the angles α, β (not demarcated in these figures) at which the screw holes extend through the plate, as described above. With reference to FIG. 12, one or more of the screw holes 20-26 can optionally be formed to extend at an angle δ with respect to the vertical direction (which is defined to be orthogonal to the top and bottom surfaces of the plate at the hole), so that the screw that is positioned in that hole can be directed into different portions of the adjacent vertebral bodies. As illustrated the example of FIG. 12, when δ is positive, the distal end of the screw for that hole would extend closer the center of curvature of the plate 10; negative values of δ would cause the distal end of that screw to extend away from the center of curvature. Preferably, −30≦δ≦+30, more preferably −20≦δ≦+20, and most preferably −15≦δ≦+15.

According to a preferred embodiment, the recessed plate and the screws are made out of titanium alloy, stainless steel, peek material, or a bioresorbable material. The plate can be any of numerous sizes; one preferred embodiment of a plate has the following dimensions: height, 9 mm-17 mm; and length, 22 mm to 36 mm; screws, diameter, from 4.0 mm to 4.5 mm and length, 20 mm-40 mm. In this context, it can be advantageous for the height of the plate 10 to be the same or slightly smaller than the height of the interbody spacer IS, so the spacer can perform its function between the two vertebrae of the patient without the plate interfering, yet the plate can still inhibit or prevent the spacer from migrating out of the intervertebral space.

According to yet another of the numerous aspects of the present invention, an exemplary method includes steps of: positioning an intervertebral spacer between two vertebrae of a patient; and positioning a plate adjacent to the intervertebral spacer and between the two vertebrae of a patient. Optionally, the plate is positioned so as to not extend beyond the exterior surface of the vertebrae, that is, the plate does not overlap onto the exterior surface of one or both of the vertebrae.

While the invention has been described in detail with reference to exemplary embodiments thereof, it will be apparent to one skilled in the art that various changes can be made, and equivalents employed, without departing from the scope of the invention. The foregoing description of the preferred embodiments of the invention has been presented for purposes of illustration and description. It is not intended to be exhaustive or to limit the invention to the precise form disclosed, and modifications and variations are possible in light of the above teachings or may be acquired from practice of the invention. The embodiments were chosen and described in order to explain the principles of the invention and its practical application to enable one skilled in the art to utilize the invention in various embodiments as are suited to the particular use contemplated. It is intended that the scope of the invention be defined by the claims appended hereto, and their equivalents. The entirety of each of the aforementioned documents is incorporated by reference herein.

What is claimed is:

1. A system comprising:
an intervertebral spacer configured and arranged to be positioned between two vertebrae of a patient; and
a plate configured and arranged to at least inhibit the intervertebral spacer from backing out when positioned between the two vertebrae of a patient.

2. A system according to claim 1, wherein the plate comprises elongate top and bottom surfaces, an elongate convex front surface extending between the top and bottom surfaces, an elongate concave rear surface extending between the top and bottom surfaces, and at least two through holes extended between the front and rear surfaces.

3. A system according to claim 2, further comprising:
at least one rotatable lock on the front surface, sized to at least partially cover the at least two holes.

4. A system according to claim 2, further comprising:
wherein at least one of the at least two holes extends at an angle toward one of the top and bottom surfaces.

5. A system according to claim 2, further comprising:
wherein the convex front surface defines a center of curvature, and wherein at least one of the at least two holes extends at an angle toward or away from the center of curvature.

6. A system according to claim 2, further comprising:
wherein the radius of curvature is about 30.5 mm.

7. A system according to claim 2, further comprising:
a bone screw positioned in each of the at least two holes.

8. A system according to claim 1, further comprising:
wherein the intervertebral spacer has a height, and wherein the distance between the top and bottom surfaces of the plate is at most the same as said height.

9. A system according to claim 1, further comprising:
wherein the intervertebral spacer is selected from the group consisting of a bone graft, a peek cage, a titanium cage, a stainless steel cage, and a bioresorbable cage.

10. A method comprising:
positioning an intervertebral spacer in an intervertebral space between two vertebrae of a patient; and
positioning a plate at least partially in said intervertebral space and adjacent to the spacer, to at least inhibit the intervertebral spacer from backing out from said space.

11. A method according to claim 10 wherein positioning a plate comprises positioning the plate entirely within the intervertebral space.

12. A method according to claim 10, wherein positioning a plate comprises securing the plate to said two vertebrae.

13. A method according to claim 10, wherein securing the plate comprises positioning at least two screws in holes in the plate and into said two vertebrae.

14. A method according to claim 10, wherein positioning at least two screws comprises positioning the screws at an angle toward one of a top and bottom surfaces of the plate.

15. A method according to claim 10, wherein the plate comprises a convex front surface with a center of curvature, and wherein positioning at least two screws comprises positioning at least one of the screws at an angle toward or away from the center of curvature.
